(19) United States
(12) Patent Application Publication
(10) Pub. No.: US 2019/0380937 A1
(43) Pub. Date: Dec. 19, 2019

(54) PROCESS FOR DEPIGMENTING KERATIN MATERIALS USING THIOPYRIDINONE COMPOUNDS

(71) Applicant: L'OREAL, Paris (FR)

(72) Inventors: Xavier MARAT, Aulnay-sous-Bois (FR); Amélie GUEGUINIAT, Aulnay-sous-Bois (FR); Sébastien GREGOIRE, Aulnay-sous-Bois (FR); Jinzhu XU, Aulnay-sous-Bois (FR); Patricio GUERREIRO, Aulnay-sous-Bois (FR)

(21) Appl. No.: 16/061,908

(22) PCT Filed: Dec. 1, 2016
(86) PCT No.: PCT/EP2016/079394

§ 371 Date: Jun. 13, 2018

(30) Foreign Application Priority Data
Dec. 18, 2015 (FR) 1562774

(51) Int. Cl. A61K 8/49; C07D 213/82; A61Q 19/02

(52) U.S. Cl. CPC A61K 8/4933; A61Q 19/02; C07D 213/82

(57) ABSTRACT

The invention relates to a cosmetic process for depigmenting, lightening and/or bleaching keratin materials, in particular the skin, comprising the application of a cosmetic composition comprising a compound of formula (I): compound of formula (I): (I) The invention also relates to the novel compounds of formula (I), to the compositions containing same and also to the cosmetic use of a compound (I) as an agent for bleaching, lightening and/or depigmenting keratin materials.

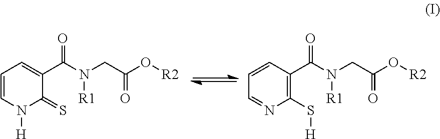

The present invention relates to a cosmetic treatment process in particular for depigmenting and/or bleaching the skin, using at least one compound of thiopyridinone type.

At various periods of their life, some people see the appearance on their skin, and more in particular on the hands, of darker and/or more coloured spots, which give the skin heterogeneity. These spots are in particular due to a high concentration of melanin in the keratinocytes located at the surface of the skin.

The use of harmless topical depigmenting substances with good efficacy is most particularly desired for the purpose of treating pigmentation spots.

The mechanism of formation of skin pigmentation, i.e. the formation of melanin, is particularly complex and schematically involves the following main steps: Tyrosine→Dopa→Dopaquinone→Dopachrome→Melanin Tyrosinase (monophenol dihydroxyl phenylalanine: oxygen oxidoreductase EC 1.14.18.1) is the essential enzyme involved in this sequence of reactions. It in particular catalyzes the reaction for conversion of tyrosine into dopa (dihydroxyphenylalanine) by means of its hydroxylase activity, and the reaction for conversion of dopa into dopaquinone by means of its oxidase activity. This tyrosinase acts only when it is in mature form under the action of certain biological factors.

A substance is acknowledged as being depigmenting if it acts directly on the vitality of the epidermal melanocytes where melanogenesis takes place, and/or if it interferes with one of the steps of melanin biosynthesis, either by inhibiting one of the enzymes involved in melanogenesis, or by inserting itself as a structural analogue of one of the chemical compounds of the melanin synthesis chain, which chain can then be blocked, thus ensuring depigmentation.

Arbutin, niacinamide and kojic acid are known as skin depigmenting agents.

Substances have been sought which have an effective depigmenting action, in particular greater than that of arbutin, niacinamide and kojic acid.

In this regard, the Applicant has discovered, surprisingly and unexpectedly, that certain thiopyridinone compounds have good depigmenting activity, even at low concentration.

A subject of the invention is thus a non-therapeutic cosmetic process for depigmenting, lightening and/or bleaching keratin materials, in particular the skin, comprising the application of a cosmetic composition comprising, in a physiologically acceptable medium, at least one compound of formula (I) as defined below.

The invention also relates to the non-therapeutic cosmetic use of a compound of formula (I) as an agent for bleaching, lightening and/or depigmenting keratin materials, in particular the skin.

The compounds used according to the invention can efficiently depigment and/or lighten, or even bleach, human skin. They are in particular intended to be applied to the skin of individuals bearing brownish pigmentation spots or senescence spots, or to the skin of individuals wishing to combat the appearance of a brownish colour caused by melanogenesis.

They may also make it possible to depigment and/or lighten bodily hairs, the eyelashes, head hair, and also the lips and/or the nails.

A subject of the invention is also the cosmetic use of a compound of formula (I) as described previously, as an agent for bleaching and/or depigmenting the skin, bodily hairs, the eyelashes or head hair, and also the lips and/or the nails, and preferably the skin, in particular for eliminating pigmentation spots or senescence spots, and/or as anti-tanning agents.

The compounds used according to the invention therefore correspond to formula (I) below:

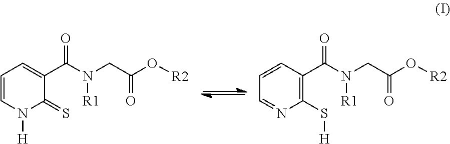

in which:
R1 denotes a radical chosen from:
a) a hydrogen atom;
b) a saturated linear C₁-C₆ alkyl group;
   R2 denotes a radical chosen from:
a) a hydrogen atom;
b) a saturated linear C₁-C₁₀ alkyl group;
c) a saturated branched C₃-C₁₀ alkyl group;
d) a C₁-C₆ phenylalkyl group such as benzyl,
   and also the tautomers thereof, the salts thereof, the solvates thereof and the optical isomers thereof, and the racemates thereof, alone or as a mixture.

The salts of the compounds of formula (I) comprise the conventional non-toxic salts of said compounds, such as those formed from an acid or base.

As salts of the compounds of formula (I), mention may be made of: the salts obtained by addition of the compound of formula (I) (when it comprises an acid group) to a mineral base, such as sodium hydroxide, potassium hydroxide, calcium hydroxide, ammonium hydroxide, magnesium hydroxide, lithium hydroxide, and sodium, potassium or calcium carbonate or hydrogen carbonate for example;

or to an organic base such as a primary, secondary or tertiary alkylamine, for example triethylamine or butylamine. This primary, secondary or tertiary alkylamine may comprise one or more nitrogen and/or oxygen atoms and may thus comprise, for example, one or more alcohol functions; mention may be made in particular of 2-amino-2-methylpropanol, ethanolamine, triethanolamine, 2-dimethylaminopropanol, 2-amino-2-(hydroxymethyl)-1,3-propanediol and 3-(dimethylamino)propylamine.

Mention may also be made of the salts of amino acids, for instance lysine, arginine, guanidine, glutamic acid and aspartic acid. Advantageously, the salts of the compounds of formula (I) (when it comprises an acid group) may be chosen from alkali metal or alkaline-earth metal salts such as sodium, potassium, calcium or magnesium salts; ammonium salts.

The acceptable solvates of the compounds described in the present invention comprise conventional solvates such as those formed during the preparation of said compounds owing to the presence of solvents. Mention may be made, by way of example, of the solvates due to the presence of water or of linear or branched alcohols, such as ethanol or isopropanol.

The optical isomers are in particular the enantiomers and the diastereoisomers.

Preferentially, the linear or branched groups may be chosen from methyl, ethyl, propyl, isopropyl, butyl, isobutyl and tert-butyl, pentyl, hexyl, heptyl, octyl, nonyl and decyl.

More preferentially, the saturated linear or branched alkyl groups may be chosen from methyl, ethyl, propyl, isopropyl, butyl, isobutyl and tert-butyl, pentyl, hexyl, heptyl and octyl.

The compound a) is disclosed in the PubCHEM database (No. 47329290) http://pubchem.ncbi.nlm.nih.gov/compound/47329290?from=summary#section=Top entry: 2010-11-26

The compound b) CAS>1240664-41-8 is described in the publication:

Synthesis of N-(2-mercaptopyridyl-3-formyl)-N-alkyl glycine and the corresponding disulfides Luo, Y. L.; Yang, Z. X.; Peng, S. X.

Div. Med. Chem., China Pharm. Univ., Nanjing, 210009, Peop. Rep. China Yaoxue Xuebao (1990), 25(5), 374-8.

Preferably, the compounds of formula (I) have the following meanings:

R1 denotes a radical chosen from:
a) a hydrogen atom;
b) a saturated linear C₁-C₄ alkyl radical and preferably methyl;

R2 denotes a radical chosen from:
a) a hydrogen atom;
b) a saturated linear C₁-C₆ alkyl group;
c) a saturated branched C₃-C₆ alkyl group,
   and also the tautomers thereof, the salts thereof, the solvates thereof and the optical isomers thereof, and the racemates thereof, alone or as a mixture.

More particularly, the compounds of formula (I) have the following meanings:

R1 denotes a radical chosen from:
a) a hydrogen atom;
b) a methyl radical;

R2 denotes a radical chosen from:
a) a hydrogen atom;
b) a saturated linear C₁-C₄ alkyl group, preferably ethyl;
c) a saturated branched C₃-C₄ alkyl group, preferably isopropyl and isobutyl,
   and also the tautomers thereof, the salts thereof, the solvates thereof and the optical isomers thereof, and the racemates thereof, alone or as a mixture.

Novel Compounds

Another subject of the invention is the novel compounds of formula (II):

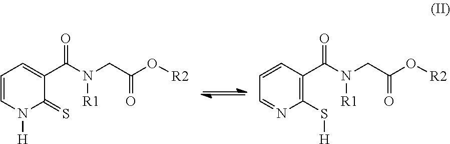

in which:

R1 denotes a radical chosen from:
a) a hydrogen atom;
b) a saturated linear C₁-C₆ alkyl group;

R2 denotes a radical chosen from:
a) a hydrogen atom;
b) a saturated linear C₁-C₁₀ alkyl group;
c) a saturated branched C₃-C₁₀ alkyl group;
d) a C₁-C₆ phenylalkyl group such as benzyl,
   and also the tautomers thereof, the salts thereof, the solvates thereof and the optical isomers thereof, and the racemates thereof, alone or as a mixture,
with the exception of the following two compounds (a) and (b) and of the tautomers thereof:

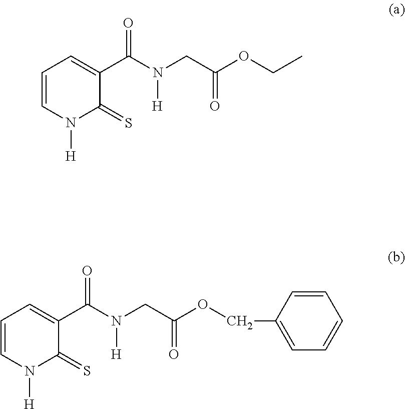 [SMILES: CCOC(=O)CNC(=O)c1ccc[nH]c1=S ; O=C(CNC(=O)c1ccc[nH]c1=S)OCc1ccccc1]

Preferably, the compounds of formula (II) have the following meanings:

R1 denotes a radical chosen from:
a) a hydrogen atom;
b) a saturated linear C₁-C₄ alkyl radical and preferably methyl;

R2 denotes a radical chosen from:
a) a hydrogen atom;
b) a saturated linear C₁-C₆ alkyl group;
c) a saturated branched C₃-C₆ alkyl group,
   and also the tautomers thereof, the salts thereof, the solvates thereof and the optical isomers thereof, and the racemates thereof, alone or as a mixture,
   with the exception of the following compound (a) and of the tautomer thereof:

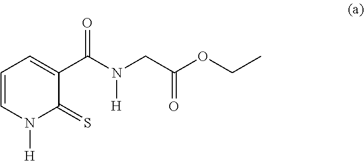 [SMILES: CCOC(=O)CNC(=O)c1ccc[nH]c1=S]

More particularly, the compounds of formula (II) have the following meanings:

R1 denotes a radical chosen from:
a) a hydrogen atom;
b) a methyl radical;

R2 denotes a radical chosen from:
a) a hydrogen atom;
b) a saturated linear C₁-C₄ alkyl group, preferably ethyl;
C) a saturated branched C₃-C₄ alkyl group, preferably isopropyl and isobutyl,
   and also the tautomers thereof, the salts thereof, the solvates thereof and the optical isomers thereof, and the racemates thereof, alone or as a mixture,
   with the exception of the following compound (a) and of the tautomer thereof:

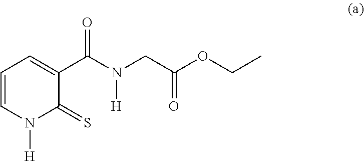 [SMILES: CCOC(=O)CNC(=O)c1ccc[nH]c1=S]

Among the compounds of formula (I) or (II), the following compounds are preferably used:

| | Compound | | |
|---|---|---|---|
| Structure | No. | Chemical name | Status |
| 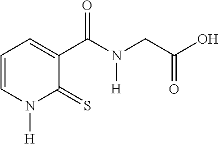 [SMILES: O=C(O)CNC(=O)c1ccc[nH]c1=S] | 1 | N-[(2-thioxo-1,2- dihydropyridin-3- yl)carbonyl]glycine | Novel product |
| 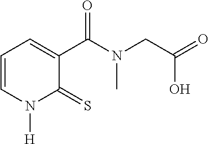 | 2 | N-methyl-N-[(2- thioxo-1,2- dihydropyridin-3- yl)carbonyl]glycine | Novel product |
| 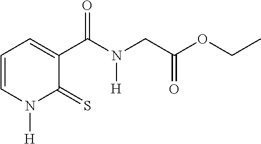 [SMILES: CCOC(=O)CNC(=O)c1ccc[nH]c1=S] | 3 | Ethyl N-[(2-thioxo- 1,2-dihydropyridin- 3-yl)carbonyl] glycinate | Compound a) |
| 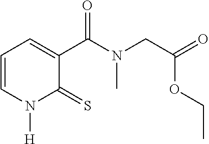 [SMILES: CCOC(=O)CN(C)C(=O)c1ccc[nH]c1=S] | 4 | Ethyl N-methyl-N- [(2-thioxo-1,2- dihydropyridin-3- yl)carbonyl] glycinate | Novel product |

and also the tautomers thereof, the salts thereof, the solvates thereof and the optical isomers thereof, and the racemates thereof, alone or as a mixture.

Among these compounds, compounds 1 and 2, and also the salts thereof, the solvates thereof, the isomers thereof and the racemates thereof, taken alone or as a mixture, are more particularly preferred.

The compounds of formula (I) can be obtained, in a known manner, by reacting 2-mercaptonicotinic acid and an amine of formula (V):

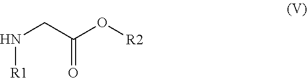

R1 and R2 having the meanings described above, in particular in the presence of a coupling agent or of a base such as carbonyldiimidazole.

The compounds of formulae (I) and (II) can also be obtained, in a known manner, by reacting 2-mercaptonicotinic acid or 2-chloronicotinic acid with an amine of formula (V) (R1 and R2 having the meanings described above), after having activated the carboxylic acid in the form of formula (W):

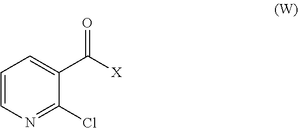

where X forms an acid halide, a mixed anhydride, a carbamimidate or an acylphosphonate by activation of the carboxylic group of the 2-chloronicotinic acid in the presence of an agent for activating carboxylic acids according to the conventional methods for activating acids (described for example in Comprehensive Organic Transformation by R. Larock, published by Wiley VCH, in the chapter Interconversion of nitriles, carboxylic acids and derivatives). Use is preferably made of an agent for activating carboxylic acids which makes it possible to form an acid chloride (for example by using thionyl or oxalyl chloride, or 1-chloro-N,N,2-trimethyl-1-propenamine) or to form a mixed anhydride (using alkyl or aryl chloroformates) or using carbodiimides or diethyl cyanophosphate to form carbamimidates or acylphosphonates (Phosphorus in organic synthesis—XI, Amino acids and peptides—XXI, Reaction of diethyl phosphorocyanidate with carboxylic acids. A new synthesis of carboxylic esters and amides, Tetrahedron, 32, 1976, 2211-2217).

When 2-chloronicotinic acid is used as starting reagent, the chloroamide of formula (Y) obtained is then used in an exchange reaction between the chlorine and the sulfur by means of reagents such as NaSH, thiourea, the Bunte salt, sodium thiosulfate or thioacetic acid (in a basic medium).

According to the overall synthesis scheme below:

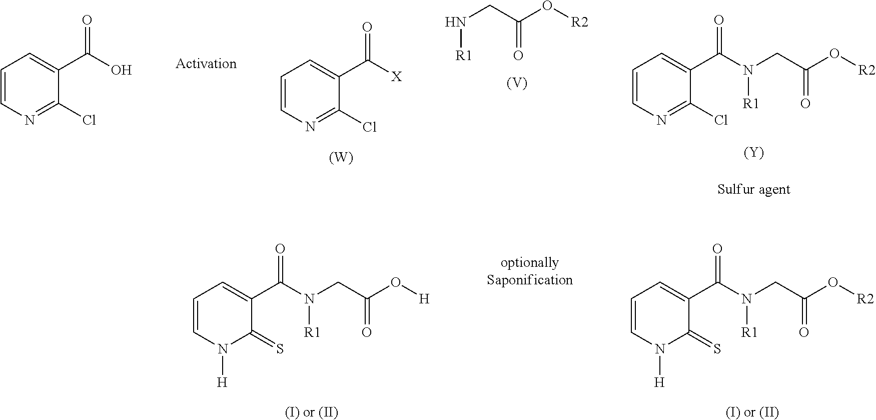

where R1, R2 and X have the same meanings as those previously defined.

In addition, when the final compounds (I) and (II) have a free carboxylic acid on the R2 radicals, said compounds can be obtained either from the corresponding amino acids or by saponification of the corresponding esters using inorganic bases such as, for example, NaOH or LiOH in the presence of protic or aprotic solvents such as, for example, ethanol or tetrahydrofuran or water, at temperatures ranging between zero and 100° C. The salts obtained can then be acidified with a mineral or organic acid such as, for example, hydrochloric acid or citric acid.

The compounds of formulae (I) and/or (II) according to the invention have a quite particular application in the cosmetics field.

The composition used according to the invention comprises a compound of formulae (I) and/or (II) as described above, in a physiologically acceptable medium.

The compound (I) and/or (II) may be present in the composition used according to the invention in an amount which may be between 0.01% and 10% by weight, preferably between 0.1% and 5% by weight, in particular from 0.5% to 3% by weight, relative to the total weight of the composition.

The term “physiologically acceptable medium” is intended to mean a medium that is compatible with human keratin materials such as the skin of the body or of the face, the lips, the mucous membranes, the eyelashes, the nails, the scalp and/or the hair.

The composition used according to the invention may then comprise any adjuvant commonly used in the cosmetics field. Mention may be made in particular of water; organic solvents, in particular C₁-C₆, more preferentially C₂-C₆, alcohols and C₂-C₁₀ carboxylic acid esters; oils, in particular hydrocarbon-based oils and/or silicone oils, of mineral, animal and/or plant origin; waxes, pigments, fillers, dyes, surfactants, emulsifiers; cosmetic or dermatological active agents, UV-screening agents, polymers, hydrophilic or lipophilic gelling agents, thickeners, preservatives, fragrances, bactericides, ceramides, odour absorbers and antioxidants.

These optional cosmetic adjuvants may be present in the composition in a proportion of from 0.001% to 80% by weight and in particular from 0.1% to 40% by weight relative to the total weight of the composition. In any case, these adjuvants, and the proportions thereof, will be chosen by those skilled in the art such that the advantageous properties of the compounds according to the invention are not, or are not substantially, adversely affected by the envisaged addition.

As active agents, it will be advantageous to introduce into the composition used according to the invention at least one compound chosen from: desquamating agents; soothing agents, organic or inorganic photo protective agents, moisturizers; depigmenting or propigmenting agents; anti-glycation agents; NO-synthase inhibitors; agents for stimulating the synthesis of dermal or epidermal macromolecules and/or for preventing their degradation; agents for stimulating fibroblast and/or keratinocyte proliferation or for stimulating keratinocyte differentiation; muscle relaxants and/or dermo-decontracting agents; tensioning agents; anti-pollution agents and/or free-radical scavengers; agents acting on the capillary circulation; agents acting on the energy metabolism of cells; and mixtures thereof.

Examples of such additional compounds are: retinol and derivatives thereof, such as retinyl palmitate; ascorbic acid and derivatives thereof, such as magnesium ascorbyl phosphate and ascorbyl glucoside; tocopherol and derivatives thereof, such as tocopheryl acetate; nicotinic acid and precursors thereof, such as nicotinamide; ubiquinone; glutathione and precursors thereof, such as L-2-oxothiazolidine-4-carboxylic acid; plant extracts, and in particular plant proteins and hydrolyzates thereof, and also phytohormones; marine extracts, such as algal extracts; bacterial extracts; sapogenins such as diosgenin and extracts of Wild Yam containing same; ceramides; hydroxy acids, such as salicylic acid and 5-n-octanoylsalicylic acid; resveratrol; oligopeptides and pseudodipeptides and acylated derivatives thereof; manganese salts and magnesium salts, in particular gluconates; and mixtures thereof.

The term “desquamating agent” is intended to mean any compound capable of acting:
either directly on desquamation by promoting exfoliation, such as β-hydroxy acids, in particular salicylic acid and derivatives thereof (including 5-n-octanoylsalicylic acid); α-hydroxy acids, such as glycolic acid, citric acid, lactic acid, tartaric acid, malic acid or mandelic acid; urea; gentisic acid; oligofucoses; cinnamic acid; extract of Saphora japonica; resveratrol;
or on the enzymes involved in the desquamation or degradation of corneodesmosomes, glycosidases, stratum corneum chymotryptic enzyme (SCCE) or other proteases (trypsin, chymotrypsin-like). Mention may be made of mineral salt chelating agents: EDTA; N-acyl-N,N′,N′-ethylenediaminetriacetic acid; aminosulfonic compounds and in particular (N-2-hydroxyethylpiperazine-N-2-ethane)sulfonic acid (HEPES); 2-oxothiazolidine-4-carboxylic acid (procysteine) derivatives; derivatives of alpha-amino acids of glycine type (as described in EP 0 852 949, and also sodium methyl glycine diacetate sold by BASF under the trade name Trilon M); honey; sugar derivatives such as O-octanoyl-6-D-maltose and N-acetylglucosamine.

The desquamating agents are generally present in the composition according to the invention in proportions ranging from 0.01% to 15% by weight, preferably ranging from 0.1% to 10% by weight relative to the total weight of the composition.

As soothing agents that can be used in the composition according to the invention, mention may be made of pentacyclic triterpenes and extracts from plants (for example Glycyrrhiza glabra) containing the same, for instance β-glycyrrhetinic acid and salts and/or derivatives thereof (glycyrrhetinic acid monoglucuronide, stearyl glycyrrhetinate, 3-stearoyloxyglycyrrhetic acid), ursolic acid and salts thereof, oleanolic acid and salts thereof, betulinic acid and salts thereof, an extract of Paeonia suffruticosa and/or lactiflora, salicylic acid salts and in particular zinc salicylate, phycosaccharides from the company Codif, an extract of Laminaria saccharina, canola oil, bisabolol and extracts of camomile, allantoin, Sepivital EPC (phosphoric diester of vitamins E and C) from SEPPIC, omega-3 unsaturated oils such as musk rose oil, blackcurrant oil, ecchium oil, fish oil, plankton extracts, capryloylglycine, Seppicalm VG (sodium palmitoylproline and Nymphea alba) from SEPPIC, an extract of Pygeum, an extract of Boswellia serrata, an extract of Centipeda cunninghami, an extract of Helianthus annuus, an extract of Linum usitatissimum, tocotrienols, extracts of Cola nitida, piperonal, an extract of clove, an extract of Epilobium angustifolium, Aloe vera, an extract of Bacopa moniera, phytosterols, cortisone, hydrocortisone, indomethacin and betamethasone.

The soothing agents are generally present in the composition used according to the invention in proportions ranging from 0.01% to 15% by weight, preferably ranging from 0.1% to 10% by weight relative to the total weight of the composition.

The organic photo protective agents are in particular chosen from anthranilates; cinnamic derivatives; dibenzoylmethane derivatives; salicylic derivatives, camphor derivatives; triazine derivatives such as those described in patent applications U.S. Pat. No. 4,367,390, EP 863 145, EP 517 104, EP 570 838, EP 796 851, EP 775 698, EP 878 469, EP 933 376, EP 507 691, EP 507 692, EP 790 243 and EP 944 624; benzophenone derivatives; β, β-diphenylacrylate derivatives; benzotriazole derivatives; benzalmalonate derivatives; benzimidazole derivatives; imidazolines; bis-benzazolyl derivatives as described in patents EP 669 323 and U.S. Pat. No. 2,463,264; p-aminobenzoic acid (PABA) derivatives; methylenebis(hydroxyphenylbenzotriazole) derivatives as described in applications U.S. Pat. Nos. 5,237,071, 5,166,355, GB 2 303 549, DE 197 26 184 and EP 893 119; screening polymers and screening silicones such as those described in particular in application WO 93/04665; α-alkylstyrene-based dimers such as those described in patent application DE 198 55 649.

The inorganic photo protective agents can in particular be chosen from coated or uncoated metal oxide pigments or nanopigments (mean size of the primary particles generally between 5 nm and 100 nm, preferably between 10 nm and 50 nm), for instance titanium oxide (amorphous or crystallized in rutile and/or anatase form), iron oxide, zinc oxide, zirconium oxide or cerium oxide nanopigments, which are all well-known UV-photoprotective agents. Conventional coating agents are, moreover, alumina and/or aluminium stearate. Such coated or uncoated metal oxide nanopigments are described in particular in patent applications EP-518 772 and EP-518 773.

The photo protective agents are generally present in the composition used according to the invention in proportions ranging from 0.1% to 20% by weight, preferably ranging from 0.2% to 15% by weight relative to the total weight of the composition.

The composition used according to the invention may be in any galenical form normally used in the cosmetics field, and in particular in the form of an aqueous or aqueous-alcoholic solution, optionally gelled, a dispersion of the lotion type, optionally a two-phase dispersion, an oil-in-water or water-in-oil or multiple (W/O/W or O/W/O for example) emulsion, an aqueous gel, a dispersion of oil in an aqueous phase by means of spherules, these spherules possibly being polymer nanoparticles such as nanospheres and nanocapsules, or, better still, lipid vesicles of ionic and/or nonionic type; aqueous or oily gel. These compositions are prepared according to the usual methods. A composition in the form of an emulsion, in particular an oil-in-water emulsion, is preferably used according to this invention.

The composition used according to the invention may constitute a skincare composition, and in particular a cleansing, protecting, treating or care cream for the face, the hands, the feet, the major anatomical folds or the body (for example day creams, night creams, makeup-removing creams, foundation creams or anti-sun creams); a fluid foundation, a makeup-removing milk, a protective or care body milk or an anti-sun milk; a skincare lotion, gel or mousse, such as a cleansing lotion.

The invention is illustrated in greater detail by the following non-limiting examples.

### EXAMPLE 1: SYNTHESIS OF COMPOUND 3—ETHYL N-[(2-THIOXO-1,2-DIHYDROPYRIDIN-3-YL)CARBONYL]GLYCINATE

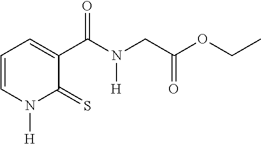 [SMILES: CCOC(=O)CNC(=O)c1ccc[nH]c1=S]

### Route 1: Via 2-mercaptonicotinic Acid

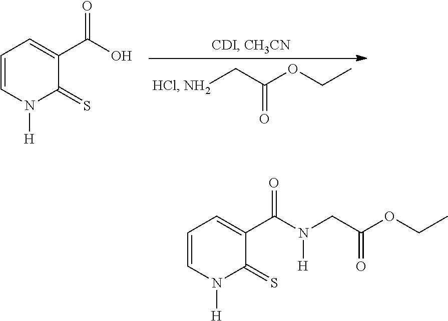

In a round-bottomed flask, thionicotinic acid is introduced into acetonitrile, followed by carbonyldiimidazole (CDI). The mixture is refluxed for 1 hour. The mixture is returned to ambient temperature and ethyl glycinate hydrochloride is added and then the heating is recommenced at 60° C. for 3 hours and then overnight at ambient temperature.

After the overnight period, the reaction medium is evaporated. The residue is taken up in dichloromethane and washed twice with 2 N HCl. The organic phase is dried over anhydrous sodium sulfate and then evaporated and purified on silica, 97/3 dichloromethane/methanol.

The fractions are evaporated.

A light-yellow-coloured powder is obtained: Yield: 37%.

### Route 2: Via 2-Chloronicotinic Acid

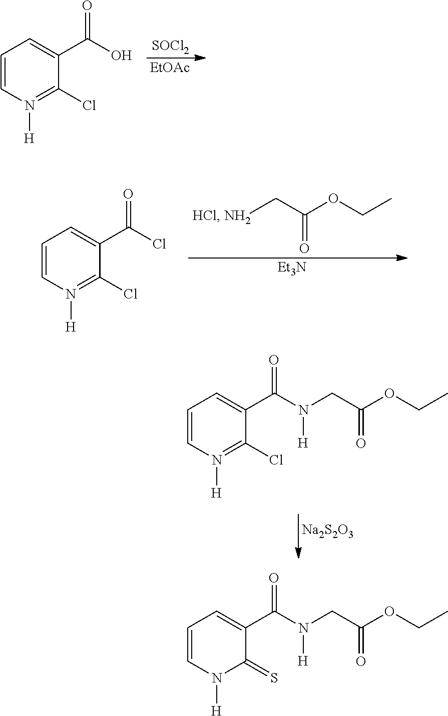

In a three-necked flask, 2-chloronicotinic acid (250.0 g, 1.587 mol) is introduced into ethyl acetate (500 ml) and SOCl₂ (210.0 g, 1.761 mol), dropwise. The mixture is refluxed until complete conversion is obtained. The medium is cooled to ambient temperature and diluted with ethyl acetate (375 ml). A solution of ethyl glycinate hydrochloride (265.8 g, 1.904 mol) diluted in water (400 ml) and triethylamine (393.5 g, 3.88 mol) are then added. The mixture is stirred for 3 to 4 h and the ethyl acetate is removed under vacuum. The aqueous solution obtained is diluted with water (1250 ml) and acidified with 3 N HCl (25 ml). Sodium thiosulfate (1379 g, 5.555 mol) is charged to the resulting solution and the mixture is refluxed for 6 h. After cooling to 10° C., the yellow solid is filtered off and washed with water (3×750 ml).

The crude product is taken up with carbon black in a 75/25 ethanol/water mixture. The carbon black is filtered off under hot conditions and the ethanol is evaporated off. The product is cooled to ambient temperature, filtered and dried under vacuum. A light-yellow-coloured powder is obtained: Yield: 88%.

The ¹H NMR and mass spectra are in accordance with the structure. Melting point: 151° C. (capillary tube)

### EXAMPLE 2: SYNTHESIS OF COMPOUND 1

### N-[(2-thioxo-1,2-dihydropyridin-3-yl)carbonyl]glycine from Compound 3

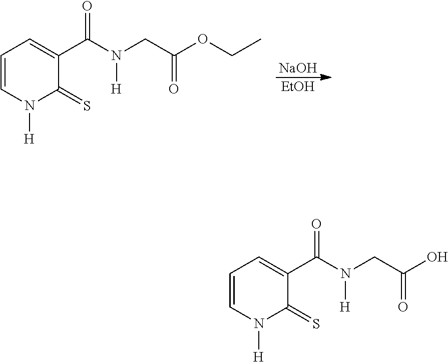

Ethyl N-[(2-thioxo-1,2-dihydropyridin-3-yl)carbonyl]glycinate (48 g, 0.317 mol), 95% EtOH (48 ml) are introduced into a three-necked flask and cooled to 10° C. A solution of NaOH (16 g) in 144 ml of water is added dropwise. The organic solvent is evaporated off and the pH is adjusted to 3-4. The resulting product is cooled to 0-10° C. and filtered. The product is washed with water and dried under vacuum.

A light-yellow-coloured powder is obtained: Yield: 96%.

The ¹H NMR and mass spectra are in accordance with the structure.

Melting point: 241.0-241.8° C. (capillary tube)

### EXAMPLE 3: SYNTHESIS OF COMPOUND 4—ETHYL N-METHYL-N-[(2-THIOXO-1,2-DIHYDROPYRIDIN-3-YL)CARBONYL]GLYCINATE

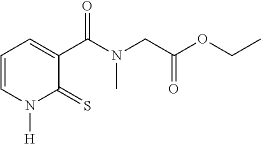 [SMILES: CCOC(=O)CN(C)C(=O)c1ccc[nH]c1=S]

### Route 1: Via 2-mercaptonicotinic Acid

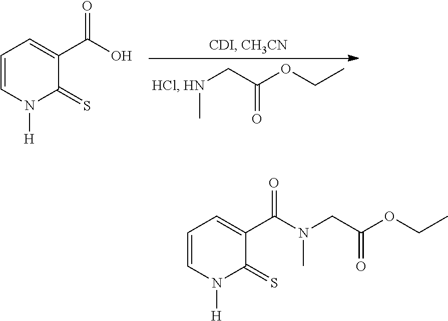

In a round-bottomed flask, thionicotinic acid is introduced into acetonitrile, followed by carbonyldiimidazole (CDI). The mixture is refluxed for 1 hour. The mixture is returned to ambient temperature and ethyl sarcosinate hydrochloride is added and then the heating is recommenced at 60° C. for 3 hours and then overnight at ambient temperature.

After the overnight period, the reaction medium is evaporated. The residue is purified on silica, 20/80 ethyl acetate/heptane.

The fractions are evaporated and then the product is taken up in dichloromethane and precipitated from isopropyl ether.

A light-yellow-coloured powder is obtained: Yield: 22%.

### Route 2: Via 2-chloronicotinic Acid

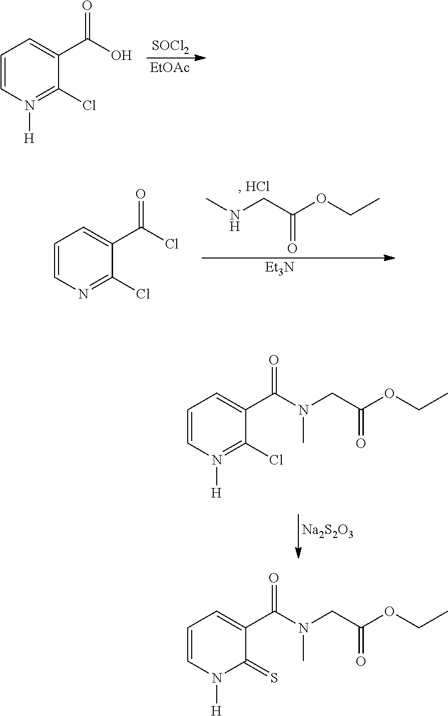

In a three-necked flask, 2-chloronicotinic acid (300.0 g, 1.904 mol) is introduced into ethyl acetate (600 ml) and SOCl₂ (249.0 g, 2.095 mol), dropwise. The mixture is refluxed until complete conversion is obtained. The medium is cooled to ambient temperature and diluted with ethyl acetate (450 ml). A solution of ethyl sarcosinate hydrochloride (351 g, 2.285 mol) diluted in water (480 ml) and triethylamine (481.8 g, 4.76 mol) are then added. The mixture is stirred for 3 to 4 h and the ethyl acetate is removed under vacuum. The aqueous solution obtained is diluted with water (1500 ml) and acidified with 3 N HCl (30 ml). Sodium thiosulfate (1796 g, 7.24 mol) is charged to the resulting solution and the mixture is refluxed until complete conversion is obtained. After cooling to 10° C., the yellow solid is filtered off and washed with water (3×900 ml). The crude product is taken up with carbon black in a 75/25 ethanol/water mixture. The carbon black is filtered off under hot conditions and the ethanol is evaporated off. The product is cooled to ambient temperature, filtered and dried under vacuum. A light-yellow-coloured powder is obtained: Yield: 69%.

The ¹H NMR and mass spectra are in accordance with the structure. Melting point: 175° C. (capillary tube)

### EXAMPLE 4: SYNTHESIS OF COMPOUND 2 FROM COMPOUND 4 N-METHYL-N-[(2-THIOXO-1,2-DIHYDROPYRIDIN-3-YL)CARBONYL]GLYCINE

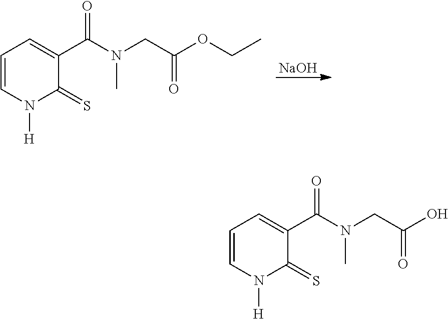

Ethyl N-methyl-N-[(2-thioxo-1,2-dihydropyridin-3-yl)carbonyl]glycinate (50 g, 0.317 mol), 95% EtOH (100 ml) and water (80 ml) are introduced into a three-necked flask. The mixture is cooled to 10° C. A solution of NaOH (23.6 g) in 70 ml of water is added dropwise. After complete conversion, the organic solvent is evaporated off and the pH is adjusted to 2-3. The resulting product is cooled to 0-10° C. and filtered. The product is washed with water and dried under vacuum. A light-yellow-coloured powder is obtained: Yield: 45%.

The ¹H NMR and mass spectra are in accordance with the structure.

Melting point: 210.9-221.8° C. (capillary tube)

### EXAMPLE 5: DEMONSTRATION OF THE ACTIVITY ON CONSTITUTIVE MELANOGENESIS

For the evaluations of the effect of prevention or of decrease of pigmentation of the skin and/or of lightening of the skin, the examples are carried out in the following way.

The measurement of the depigmenting activity (reduction of melanin production) of compounds of formula (I) was carried out by assaying normal human melanocytes in vitro as follows.

First of all, normal human melanocytes are cultured and dispensed into 384 wells. After 24 hours, the culture medium was replaced with a medium containing compounds of formula (I) to be evaluated. The cells were incubated for 72 hours before measuring the final optical density, which measures the amount of melanin produced by the melanocytes. A dose-effect is performed using a wide concentration range of the compounds evaluated. Thus, by making the concentrations and the melanin measurements correspond, it is possible to determine an IC50 in μM: concentration at which 50% decrease in melanin synthesis is achieved. The highest concentration used in the test is 200 μM.

The compounds of formula (I) showed a strong depigmenting effect.

| Compound No. | IC50 (μM) |
|---|---|
| 1 | 11.4 |
| 2 | 5.27 |
| 3 | 36.8 |
| 4 | 67.6 |

In another campaign, compound 3 was compared with the closest compound of the prior art described in patent FR 2 968 661:

| Compound No. | IC50 (μM) |
|---|---|
| 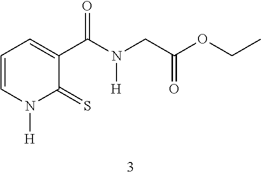 [SMILES: CCOC(=O)CNC(=O)c1ccc[nH]c1=S] | 20.8 |
| 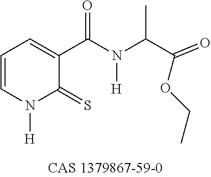 [SMILES: CCOC(=O)C(C)NC(=O)c1ccc[nH]c1=S] | 37.4 |

The compounds of the invention have an activity on melanogenesis reduction that is greater than that of the compound (CAS 1379867-59-0) outside the invention.

### EXAMPLE 6: COSMETIC COMPOSITION

A skin depigmenting composition is prepared, comprising (in grams):

| | |
|---|---|
| Compound No. 4 | 2 g |
| PEG 400 | 68 g |
| Ethanol | 30 g |

The composition applied to the skin makes it possible to cause brown spots to become less marked.

### EXAMPLE 7: GEL

A skin depigmenting gel is prepared, comprising (% by weight):

| | |
|---|---|
| Compound No. 1 | 0.5% |
| Carbomer | 1% |
| (Carbopol 981 from Lubrizol) | |
| preservative | qs |
| water | qs 100% |

The composition applied to the skin makes it possible to cause brown spots to become less marked.

### EXAMPLE 8

### Test on Pigmented Reconstructed Epidermis Samples

Compositions comprising 300 μM of compound 1, 2, 3 or 4 in DMSO were applied to samples of pigmented reconstructed epidermis (cf. EP 1 878 790). The control is DMSO. The melanin was quantified by image analysis on histological slices after staining with Fontana Masson dye. Each sample of coloured epidermis is photographed over its entire length using a camera connected to a microscope. The melanin is thresholded and the number of melanin pixels is measured in each field using automated image analysis software. A non-parametric statistical test is performed in order to determine the significance of the measurements (Mann-Whitney test).

The pigmented reconstructed epidermis standard study model was published by:

Regnier M, Duval C, Galey J B, Philippe M, Lagrange A, Tuloup R, Schmidt R, Cellular and Molecular Biology, 1999, 45, 7, 969-980: “Keratinocyte-Melanocyte co-cultures and pigmented reconstructed human epidermis: models to study modulation of melanogenesis”.

Significant depigmenting activity was evaluated at 100 μM for compounds 1 and 2. (pValue<0.05: significant depigmenting activity).

Significant depigmenting activity was evaluated at 300 μM for compounds 3 and 4. (pValue<0.05: significant depigmenting activity).

1.-16. (canceled)

17. A method for depigmenting, lightening, and/or bleaching keratin materials, comprising applying to the keratin materials, a cosmetic composition, the cosmetic composition comprising, in a physiologically acceptable medium, at least one compound of formula (I): 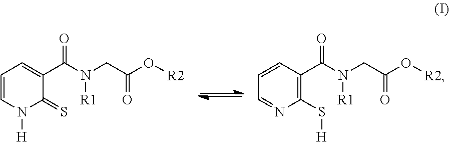
wherein:
R1 is a radical chosen from:
a) a hydrogen atom; or
b) a saturated linear C₁-C₆ alkyl group; and
R2 is a radical chosen from:
a) a hydrogen atom;
b) a saturated linear C₁-C₁₀ alkyl group;
c) a saturated branched C₃-C₁₀ alkyl group; or
d) a C₁-C₆ phenylalkyl group,
or a tautomer thereof, a salt thereof, a solvate thereof, an optical isomer thereof, a racemate thereof, or mixtures thereof.

18. The method of claim 17, wherein:
R1 is a radical chosen from:
a) a hydrogen atom; or
b) a saturated linear C₁-C₄ alkyl radical; and
R2 is a radical chosen from:
a) a hydrogen atom;
b) a saturated linear C₁-C₆ alkyl group; or
c) a saturated branched C₃-C₆ alkyl group.

19. The method of claim 17, wherein:
R1 is a radical chosen from:
a) a hydrogen atom; or
b) a methyl radical; and
R2 is a radical chosen from:
a) a hydrogen atom;
b) a saturated linear C₁-C₄ alkyl group; or
c) a saturated branched C₃-C₄ alkyl group.

20. The method of claim 17, wherein the at least one compound of formula (I) is chosen from:

| | Compound | |
|---|---|---|
| Structure | No. | Chemical name |
| 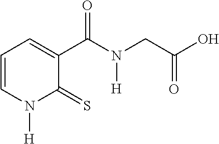 [SMILES: O=C(O)CNC(=O)c1ccc[nH]c1=S] | 1 | N-[(2-thioxo-1,2-dihydropyridin-3- yl)carbonyl]glycine |
| 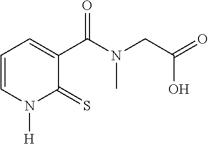 | 2 | N-methyl-N-[(2-thioxo-1,2- dihydropyridin-3- yl)carbonyl]glycine |
| 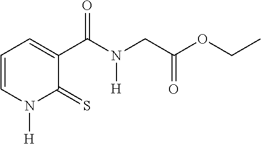 [SMILES: CCOC(=O)CNC(=O)c1ccc[nH]c1=S] | 3 | Ethyl N-[(2-thioxo-1,2- dihydropyridin-3- yl)carbonyl]glycinate |
| 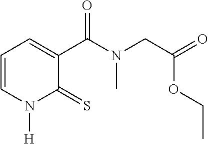 [SMILES: CCOC(=O)CN(C)C(=O)c1ccc[nH]c1=S] | 4 | Ethyl N-methyl-N-[(2-thioxo-1,2- dihydropyridin-3- yl)carbonyl]glycinate |

or a tautomer thereof, a salt thereof, a solvate thereof, an optical isomer thereof, a racemate thereof, or mixtures thereof.

21. The method of claim 17, wherein the at least one compound of formula (I) is chosen from:

| | Compound | |
|---|---|---|
| Structure | No. | Chemical name |
| 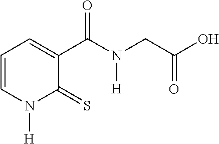 [SMILES: O=C(O)CNC(=O)c1ccc[nH]c1=S] | 1 | N-[(2-thioxo-1,2- dihydropyridin-3- yl)carbonyl]glycine |
| 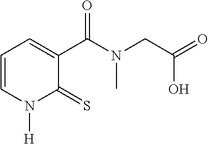 | 2 | N-methyl-N-[(2-thioxo-1,2- dihydropyridin-3- yl)carbonyl]glycine |

22. The method of claim 17, wherein the at least one compound of formula (I) is present in an amount ranging from about 0.01% to about 10% by weight, relative to the total weight of the composition.

23. The method of claim 17, wherein the at least one compound of formula (I) is present in an amount ranging from about 0.5% to about 3% by weight, relative to the total weight of the composition.

24. The method of claim 17, wherein the composition further comprises at least one adjuvant chosen from water; organic solvents; carbon-based or silicone oils of mineral, animal, or plant origin; waxes, pigments; fillers; dyes; surfactants; emulsifiers; co-emulsifiers; cosmetic or dermatological active agents; UV screening agents; polymers; hydrophilic or lipophilic gelling agents; thickeners; preservatives; fragrances; bactericides; ceramides; odor absorbers; antioxidants; or mixtures thereof.

25. The method of claim 17, wherein the composition comprises at least one active agent chosen from desquamating agents; soothing agents; organic or inorganic photo protective agents; moisturizers; depigmenting or propigmenting agents; anti-glycation agents; NO-synthase inhibitors; agents for stimulating the synthesis of dermal or epidermal macromolecules and/or for preventing their degradation; agents for stimulating fibroblast and/or keratinocyte proliferation or for stimulating keratinocyte differentiation; muscle relaxants and/or dermo-decontracting agents; tensioning agents; anti-pollution agents and/or free-radical scavengers; agents acting on the capillary circulation; agents acting on the energy metabolism of cells; or mixtures thereof.

26. A compound of formula (II): 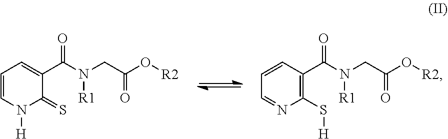
wherein:
R1 is a radical chosen from:
a) a hydrogen atom; or
b) a saturated linear C₁-C₆ alkyl group;
R2 is a radical chosen from:
a) a hydrogen atom;
b) a saturated linear C₁-C₁₀ alkyl group;
c) a saturated branched C₃-C₁₀ alkyl group; or
d) a C₁-C₆ phenylalkyl group,
or a tautomer thereof, a salt thereof, a solvate thereof, an optical isomer thereof, a racemate thereof, or mixtures thereof,
with the exception of the following two compounds (a) and (b) and the tautomers thereof:

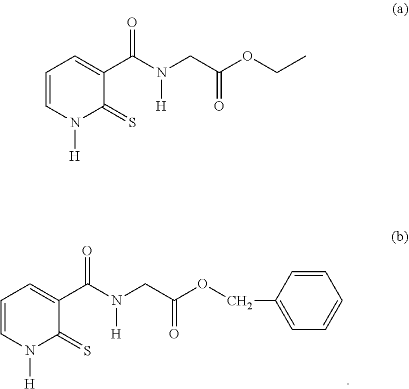 [SMILES: CCOC(=O)CNC(=O)c1ccc[nH]c1=S ; O=C(CNC(=O)c1ccc[nH]c1=S)OCc1ccccc1]

27. The compound of claim 26, wherein:
R1 is a radical chosen from:
a) a hydrogen atom; or
b) a saturated linear C₁-C₄ alkyl radical;
R2 is a radical chosen from:
a) a hydrogen atom;
b) a saturated linear C₁-C₆ alkyl group; or
c) a saturated branched C₃-C₆ alkyl group,
or a tautomer thereof, a salt thereof, a solvate thereof, an optical isomer thereof, a racemate thereof, or mixtures thereof,
with the exception of the following compound (a) and the tautomers thereof:

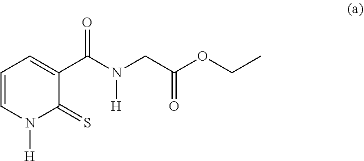 [SMILES: CCOC(=O)CNC(=O)c1ccc[nH]c1=S]

28. The compound of claim 26, chosen from:

| | Compound | |
|---|---|---|
| Structure | No. | Chemical name |
| 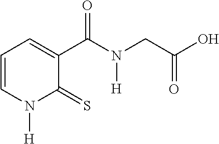 [SMILES: O=C(O)CNC(=O)c1ccc[nH]c1=S] | 1 | N-[(2-thioxo-1,2- dihydropyridin-3- yl)carbonyl]glycine |
| 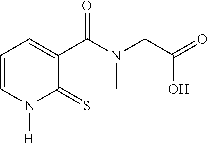 | 2 | N-methyl-N-[(2-thioxo-1,2- dihydropyridin-3- yl)carbonyl]glycine |
| 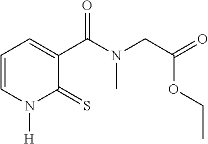 [SMILES: CCOC(=O)CN(C)C(=O)c1ccc[nH]c1=S] | 4 | ethyl N-methyl-N-[(2- thioxo-1,2-dihydropyridin-3- yl)carbonyl]glycinate |

or a tautomer thereof, a salt thereof, a solvate thereof, an optical isomer thereof, a racemate thereof, or mixtures thereof.

29. A cosmetic composition comprising at least one compound of formula (II), wherein:
R1 is a radical chosen from:
a) a hydrogen atom; or
b) a saturated linear C₁-C₆ alkyl group;
R2 is a radical chosen from:
a) a hydrogen atom;
b) a saturated linear C₁-C₁₀ alkyl group;
c) a saturated branched C₃-C₁₀ alkyl group; or
d) a C₁-C₆ phenylalkyl group,
or a tautomer thereof, a salt thereof, a solvate thereof, an optical isomer thereof, a racemate thereof, or mixtures thereof,
with the exception of the following two compounds (a) and (b) and the tautomers thereof:

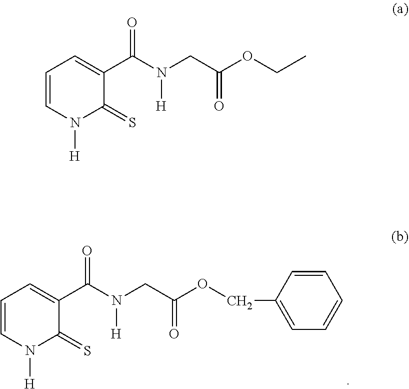 [SMILES: CCOC(=O)CNC(=O)c1ccc[nH]c1=S ; O=C(CNC(=O)c1ccc[nH]c1=S)OCc1ccccc1]

30. The composition of claim 29, wherein the at least one compound of formula (II) is present in an amount ranging from about 0.01% to about and 10% by weight, relative to the total weight of the composition.

31. The composition of claim 29, wherein the at least one compound of formula (II) is present in an amount ranging from about 0.5% to about 3% by weight, relative to the total weight of the composition.

32. A method for preparing compounds of formulae (I) and (II) below: 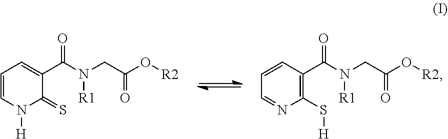
wherein:
R1 is a radical chosen from:
a) a hydrogen atom; or
b) a saturated linear C₁-C₆ alkyl group; and
R2 is a radical chosen from:
a) a hydrogen atom;
b) a saturated linear C₁-C₁₀ alkyl group;
c) a saturated branched C₃-C₁₀ alkyl group; or
d) a C₁-C₆ phenylalkyl group,
or a tautomer thereof, a salt thereof, a solvate thereof, an optical isomer thereof, a racemate thereof, or mixtures thereof; 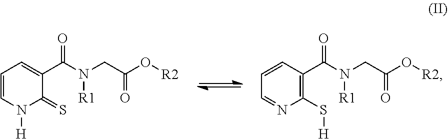
wherein:
R1 is a radical chosen from:
a) a hydrogen atom; or
b) a saturated linear C₁-C₆ alkyl group;
R2 is a radical chosen from:
a) a hydrogen atom;
b) a saturated linear C₁-C₁₀ alkyl group;
c) a saturated branched C₃-C₁₀ alkyl group; or
d) a C₁-C₆ phenylalkyl group,
or a tautomer thereof, a salt thereof, a solvate thereof, an optical isomer thereof, a racemate thereof, or mixtures thereof,
with the exception of the following two compounds (a) and (b) and the tautomers thereof: 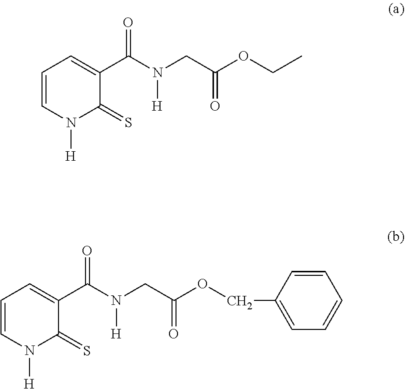 [SMILES: CCOC(=O)CNC(=O)c1ccc[nH]c1=S ; O=C(CNC(=O)c1ccc[nH]c1=S)OCc1ccccc1]
according to the following scheme: 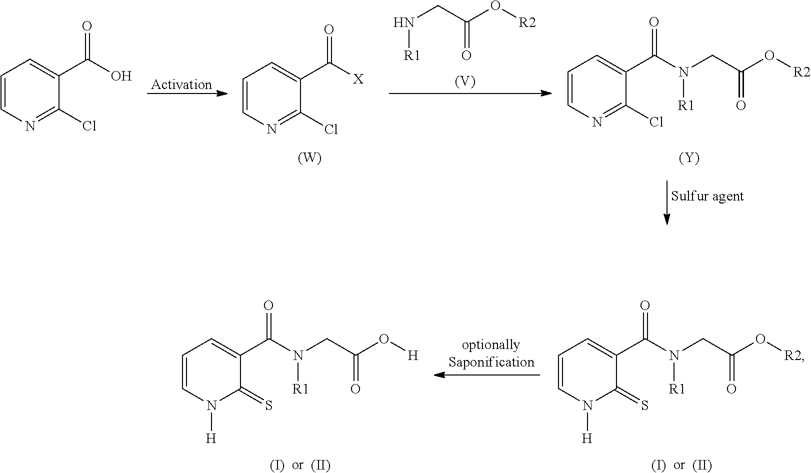
wherein:
R1 is a radical chosen from:
a) a hydrogen atom; or
b) a saturated linear C₁-C₆ alkyl group;
R2 is a radical chosen from:
a) a hydrogen atom;
b) a saturated linear C₁-C₁₀ alkyl group;
c) a saturated branched C₃-C₁₀ alkyl group; or
d) a C₁-C₆ phenylalkyl group, and
X forms an acid halide, a mixed anhydride, a carbamimidate, or an acylphosphonate by activation of the carboxylic group of the 2-chloronicotinic acid in the presence of an agent for activating carboxylic acids according to the conventional methods for activating acids.

33. The method of claim 32, comprising:
(i) preparing a compound of formula (W): 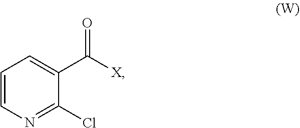
wherein X forms an acid halide, a mixed anhydride, a carbamimidate, or an acylphosphonate by activation of the carboxylic group of the 2-chloronicotinic acid in the presence of an agent for activating carboxylic acids according to the conventional methods for activating acids;
ii) reacting the compound (W) with an amine of formula (V): 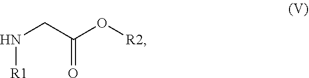
wherein:
R1 is a radical chosen from:
a) a hydrogen atom; or
b) a saturated linear C₁-C₆ alkyl group; and
R2 is a radical chosen from:
a) a hydrogen atom;
b) a saturated linear C₁-C₁₀ alkyl group;
c) a saturated branched C₃-C₁₀ alkyl group; or
d) a C₁-C₆ phenylalkyl group,
so as to form a compound of formula (Y); 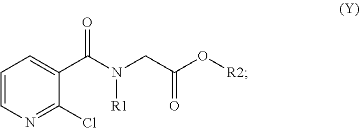
and
iii) exchanging between the chlorine and the sulfur by means of reagents so as to form the compounds of formulae (I) or (II),
iv) optionally, for the compounds of formulae (I) and (II) wherein the R2 radical is a hydrogen atom, the compounds are obtained either directly or by means of an additional step of saponification of the corresponding esters using inorganic bases following acidification.
